(19) United States
(12) Patent Application Publication
(10) Pub. No.: US 2003/0143199 A1
(43) Pub. Date: Jul. 31, 2003

(54) Use of STAT-6 inhibitors as therapeutic agents

(76) Inventors: Dennis A. Carson, Del Mar, CA (US); Howard B. Cottam, Escondido, CA (US); Lorenzo M. Leoni, San Diego, CA (US); Sylvie Barchechath, La Jolla, CA (US)

Correspondence Address: SCHWEGMAN, LUNDBERG, WOESSNER & KLUTH, P.A., P.O. BOX 2938, MINNEAPOLIS, MN, 55402, US

(21) Appl. No.: 10/269,110

(22) Filed: Oct. 9, 2002

Related U.S. Application Data
(60) Provisional application No. 60/328,162, filed on Oct. 9, 2001.
(60) Provisional application No. 60/328,689, filed on Oct. 10, 2001.

(51) Int. Cl. A61K 38/21; A61K 31/522; A61K 31/429; A61K 31/424; A61K 31/4184; A61K 31/7072; A61K 31/7076

(52) U.S. Cl. 424/85.7; 514/368; 514/375; 514/393; 514/359; 514/50; 514/43; 514/263.35

(57) ABSTRACT

The present invention provides A therapeutic method to enhance the efficacy of interferon treatment comprising administering to a mammal subject to interferon treatment a compound which is an antagonist of the IL-4 or IL-13 signal transduction pathway in an amount effective to enhance said efficacy. The method includes treatment of diseases such as cancer, proliferative fibrotic diseases, viral diseases, or autoimmune diseases. The invention also includes the use of chemotherapeutic agents, radiation or other treatments in conjunction with the method of the invention.

### CROSS-REFERENCE TO RELATED APPLICATIONS

This application claims priority of U.S. provisional patent application Serial No. 60/328,162, filed Oct. 9, 2001, and U.S. provisional patent application Serial No. 60/328,689, filed Oct. 10, 2001, both of which are incorporated by reference herein.

### BACKGROUND OF THE INVENTION

This invention was made with the assistance of the National Institutes of Health under Grant Nos. GM23200 and CA81534. The U.S. Government has certain rights in this invention.

The cytokines IL-4 and IL-13 interact with receptors on target B cells, and stimulate the production of IgE and other mediators of allergy. However, recent data indicate that IL-4/IL-13 signaling also (1) inhibits apoptosis in malignant B cells and other cancer cells, (2) prevents the rejection of tumors by the body, (3) promotes the survival of fibroblasts and therefore increases fibrosis, and (4) stimulates the differentiation of antigen-presenting cells.

The STAT4 and STAT6 genes encode transcription factors that when phosphorylated by Janus kinases are activated and transported to the nucleus where they regulate cytokine-induced gene expression. See, e.g., J. T. Ihle, Stem Cells Suppl., 1, 105 (1997); M. Heim, J. Recept. Signal. Transduction Res., 19, 75 (1999); K. S. Liu et al., Curr. Opin. Immunol., 10, 271 (1998). For example, STAT-6 is the common transcription factor for IL-4 and IL-13.

STAT4 and STAT6 are essential for the development of CD4⁺ Th1 and Th2 development, respectively. Tumor immunologists have hypothesized that Th1 cells are critical in tumor immunity because they facilitate differentiation of CD8⁺ T cells, which are potent anti-tumor effectors. S. Ostrand-Rosenberg et al., J. Immunol., 165, 6015 (2000) used STAT4^{-/-} and STAT6^{-/-} mice to test this hypothesis. BALB/c and knockout mice were challenged in the mammary gland with the highly malignant and spontaneously metastatic BALB/c-derived 4T1 mammary carcinoma. Primary tumor growth and metastatic disease were reduced in STAT6^{-/-} mice relative to BALB/c and STAT4^{-/-} mice. Ab depletions demonstrated that the effect is mediated by CD8⁺ T cells, and immunized STAT6^{-/-} mice had higher levels of 4T1-specific CTL than BALB/c or STAT4^{-/-} mice. Th1 or Th2 cells were not involved, because CD4 depletion did not diminish the anti-tumor effect. Therefore, deletion of the STAT6 gene facilitates development of potent anti-tumor immunity via a CD4⁺-independent pathway.

Sumitumo Pharmaceutical Co. (published Japanese Patent Application, JP 1997/000288026) discloses certain imidazo [2,1-b]thiazole derivatives that are capable of inhibiting STAT-6. The compounds are disclosed to be useful for the treatment and prevention of allergic diseases and parasitic infectious diseases. However, a continuing need exists for small molecules that can inhibit STAT-6 and thus, inhibit IL-4 and IL-13 signal transduction. Such compounds can be used therapeutically as discussed hereinbelow.

In addition, there is a need for novel, potent, and selective agents to prevent detrimental effects upon cells due to DNA damage, such as caused by chemotherapy, radiation, ischemic event, including ischemia-reperfusion injury and organ transplantation, and the like. There is also a need for pharmacological tools for the further study of the physiological processes associated with intracellular DNA damage.

p53, the product of the p53 tumor suppressor gene, is a multifunctional tumor suppressor protein, involved in the negative control of cell growth. In response to a variety of stressors, p53 induces growth arrest or apoptosis, thereby eliminating damaged and potentially dangerous cells. T. M. Gottleib et al., Biochim. Biophys. Acta, 1287, 77 (1996). Mutations in the p53 gene are frequently associated with the metastatic stage of tumor progression, and lack of functional p53 is accompanied by rapid tumor progression, resistance to anti-cancer therapy and increased tumor angiogenesis. See, e.g., A. J. Levine et al., Br. J. Cancer, 69, 409 (1994); R. J. Steele et al., Br. J. Surg., 85, 1460 (1998); C. Cordon-Cardo et al., Surg. Oncol., 13, 319 (1997). p53 deficiency in mice is associated with a high frequency of spontaneous cancers. L. A. Donehower et al., Nature, 356, 215 (1992); T. Jacks et al., Curr. Biol., 4, 1 (1994). On the basis of these reports, the inactivation of p53 was viewed as an unfavorable event, and it has been speculated that cancer can be inhibited by restoration of p53 function.

A continuing need exists for compounds that can protect mammalian cells from the damaging effects of chemotherapy and irradiation, or in other situations in which it is desirable to protect tissue from the consequences of clinical or environmental stress.

### SUMMARY OF THE INVENTION

The present invention provides compounds that act to inhibit the activity of STAT-6 in mammalian cells, and a method to effectively inhibit signal transduction through the IL-4 and IL-13 pathways, in vitro or in vivo, in the cells of a mammal, such as a human, subject to pathology that is ameliorated by such inhibition. Accordingly, there is provided a method of suppression comprising administering to a mammal in need of said suppression an effective amount of a compound of formula (I):

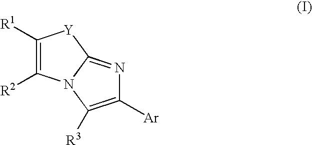

wherein R¹, R² and R³ are independently hydrogen, halo, hydroxy, cyano, —N(Rₐ)(R_{b}), —S(Rₐ), —NO₂, (C₁-C₆)alkyl, (C₁-C₆)alkoxy, (C₂-C₆)alkynyl, (C₂-C₆)-alkenyl, (C₂-C₇)alkanoyl, (C₂-C₇)alkanoyloxy, or (C₃-C₇)cycloalkyl or R¹ and R² taken together are benzo, optionally substituted by R¹, or are (C₃-C₅)alkylene or methylenedioxy; wherein Rₐ and R_{b} are each independently hydrogen, (C₁-C₃)alkyl, (C₂-C₄)alkanoyl, phenyl, benzyl, or phenethyl; or Rₐ and R_{b} together with the nitrogen to which they are attached are a 5-6 membered heterocyclic ring, preferably a pyrrolidino, piperidino or morpholino ring;

Ar is aryl, heteroaryl, or a 5-6 membered heterocyclic ring, preferably comprising 1-3 N(Rₐ), nonperoxide O or S atoms, such as a pyrrolidino, piperidino or morpholino ring, optionally substituted with 1-5, preferably 1-2, halo, CF₃, hydroxy, CN, —N(Rₐ)(R_{b}), (C₁-C₆)alkyl, (C₁-C₆)alkoxy, (C₂-C₇)alkanoyl, (C₂-C₇)-alkanoyloxy, (C₃-C₇)cycloalkyl, (C₂-C₆)alkenyl, or phenyl groups;

Y is oxy (—O—), —S(O)₀₋₂-, Se, —C(R¹)(R³)—, —N(Rₐ)—, or —P—;

or a pharmaceutically acceptable salt thereof.

Preferably, Ar is not substituted with halo or alkoxy. Preferably, Ar is heteroaryl or a heterocyclic ring. Preferably, R¹ and R² are not benzo or (C₃-C₅)alkylidenyl when Ar is aryl, e.g., is phenyl or napthyl. Novel compounds of formula (I) are also within the scope of the present invention, e.g., preferably Y is —O—, —Se—, —C(R₁)(R₃)—, or P. Preferably, Ar is heteroaryl. Preferably, Ar is substituted with CN, (C₂-C₇)alkanoyl), (C₂-C₇)alkanoyloxy, (C₃-C₇)cycloalkyl, (C₂-C₆)alkenyl or combinations thereof. Preferably, R¹, R² and R³ are independently, OH, CN, —N(Rₐ)(R_{b}), —S(Rₐ), —NO₂, (C₂-C₇)alkanoyl, or (C₂-C₇)-alkanoyloxyl.

The present method also provides a therapeutic method comprising suppressing STAT-6 or the IL-4/IL-13 pathways in mammalian cells in vitro or in vivo, and thus treating a pathological condition ameliorated by said suppression, comprising administering to a mammal in need of said suppression an effective amount of a compound of formula (II):

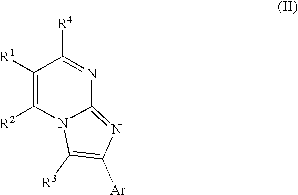

wherein R₁, R₂ and R₃ as well as Ar are defined as above; R₄ is the same as, but independent from, R₁, R₂ and R₃. R₄ in combination with R₁ can also be benzo, (C₃-C₅)alkylidene or methylenedioxy. These compounds are imidazo[1,2-a]-quinazolines.

Compounds of formula (II) also include (IIa) and (IIb):

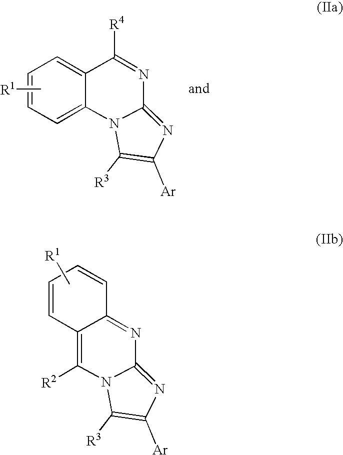

wherein R₁, R₂, R₃ and R₄ are as defined herein. Novel compounds of formulae II, IIa and IIb are also within the scope of the invention. Preferably, R₄ is not OH in IIa or IIb, e.g., where R₁ and R₂ or R₁ and R₄ are benzo. In compounds of formula II, R₁ and R₂ are preferably not benzo when Ar is phenyl.

The present invention also includes compounds of formula III:

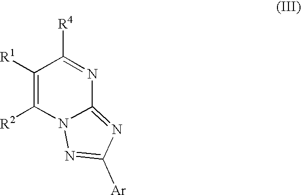

wherein R₁, R₂ and R₄, as well as Ar are defined as herein, for formula (I).

Also included within the invention are methods of using compounds of formula III in amounts effective to suppress STAT-6 or the IL-4/IL-13 pathways in mammalian cells, and thus to provide treatment for a mammal afflicted by a pathology ameliorated by said suppression.

Compounds of formula (IV) are also included in the invention:

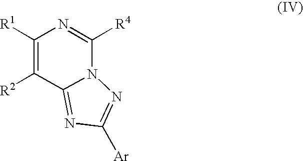

wherein R₁, R₂ and R₄, as well as Ar are defined as above, for formula (II), as well as methods for their use to treat conditions ameliorated by a suppression of STAT-6 or by inhibition of signal transduction through the IL-4/IL-13 pathways in mammalian cells in vitro or in vivo. Preferably, R₁ and R₂ are not benzo when R₄ is H or OH.

Compounds of formula (V) are also included in the invention:

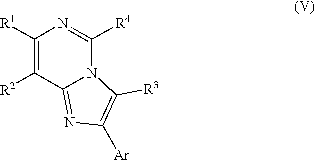

wherein R₁, R₂, R₃ and R₄ as well as Ar are defined as above, for formula (II), as well as methods for their use as discussed above. Preferably, Ar is not 4-methoxyphenyl when R₁ and R₂ are benzo and R₄ is H.

Compounds of formulae (I)-(V) are small molecule antagonists of IL-4/IL-13 signal transduction in mammalian cells in vitro and in vivo. These molecules can inhibit the survival of malignant B cells and sensitize them to other chemotherapeutic agents, such as, for example, interferons, particularly α-interferon, β-interferon and γ-interferon. These compounds are relatively nontoxic to normal lymphocytes. Antibodies to IL-4 and IL-13 receptors and to other receptors are in clinical trials. However, IL-4 and IL-13 have redundant activities, and thus blocking only one of them is insufficient in many instances. Preferred compounds (I)-(IV) can block both IL-4 and IL-13 signaling. They may act by inhibiting expression of the STAT-6 gene, and thus by inhibiting STAT-6, the common transcription factor for IL-4 and IL-13. They can be useful to treat cancer, fibrotic diseases and inflammatory diseases.

More specifically, compounds (I)-(V) may be useful for:

1. Treatment of leukemia, lymphoma, Hodgkin's, lung, head, neck, glioblastomas and other cancers expressing IL-4 and/or IL-13 receptors (e.g., gliomas and head and neck cancers).

2. Sensitization of cancer cells to monoclonal antibodies and chemotherapeutic agents.

3. Use in vaccines against cancer and viral diseases to increase cytotoxic T cell responses.

4. Treatment of proliferative fibrotic diseases, such as rheumatoid arthritis, pulmonary fibrosis, liver cirrhosis, and chronic kidney diseases.

5. Use alone or in combination with therapeutic agents for treatment of viral diseases such as hepatitis, papilloma or RNA viruses such as Semliki Forest virus, San Angelo Virus, Punta Toro virus, Banzi virus and the like.

6. Use alone or in combination with therapeutic agents for treatment of autoimmune diseases such as autoimmune diseases, such as lupus erythematosus, multiple sclerosis, infertility from endometriosis, type I diabetes mellitus, Crohn's disease, ulcerative colitis, inflammatory bowel disease, and rheumatoid arthritis or diseases such as pulmonary fibrosis.

IL-4 and IL-13 are known to be essential for asthma and allergies. T. Akimoto et al., J. Exp. Med., 182, 1537 (1998) report that STAT-6 deficient mice, which cannot respond to IL-4/IL-13, also do not develop allergic asthma.

M. Dancescu et al., J. Exp. Med., 176, 1319 (1992) and U. Kapp, J. Exp. Med., 189, 1939 (1999) report that IL-4 and IL-13 are survival factors for malignant cells in chronic lymphocytic leukemia and Hodgkin's disease (a form of lymphoma). Thus, the present compounds should be useful for treatment of these diseases.

K. Kawakami et al., Cancer Res., 60, 2981 (2000) reports the expression of IL-4 receptors in head and neck cancer, melanoma, breast cancer, ovary cancer, neuroblastomas, renal carcinomas. The present compounds thus can be useful for treatment of these cancers.

M. Terabe et al., Nature/Immunol., 1, 516 (2000) and S. Ostrand-Rosenberg, cited above, report the remarkable finding that lack of STAT-6 signaling promoters immune rejection of cancers. Thus, the claimed compounds can be used in cancer vaccines and/or with monoclonal antibodies to enhance their immunologic effects.

U. Muller-Ladner et al., J. Immunol., 164, 3894 (2000) reported that the IL-4 pathway is active in the fibroblasts that show unrestrained growth in the joints of patients with rheumatoid arthritis. Similar outgrowth of fibroblasts is seen in pulmonary fibrosis, cirrhosis, renal diseases, scleroderma. The present compounds can be useful in all these conditions.

The invention also provides pharmaceutical compositions comprising novel compounds of formula (I)-(V), or a pharmaceutically acceptable salt thereof, in combination with a pharmaceutically acceptable diluent or carrier.

The invention also provides novel compounds of formula (I), or a pharmaceutically acceptable salt thereof, in combination with a pharmaceutically acceptable diluent or carrier. Such compounds can be represented by compounds of formula (I), with the proviso that when Y is S, Ar is not phenyl (C₆H₅).

Additionally, the invention provides a therapeutic method for preventing or treating a pathological condition or symptom in a mammal, such as a human, wherein the activity of STAT-6 or IL-4/IL-13-mediated signal transduction is implicated and antagonism or suppression of their action is desired, comprising administering to a mammal in need of such therapy, an effective amount of one or more compounds of formula (I)-(V), or a pharmaceutically acceptable salt thereof. Such pathological conditions or symptoms include treatment of cancers expressing IL-4 and/or IL-13 receptors, sensitization of cancer cells to chemotherapy or radiation, increasing T_{c} cell responses and the treatment of proliferative fibrotic disease.

The invention provides a compound of formula (I)-(V) for use in medical therapy as well as the use of a compound of formula (I)-(V) for the manufacture of a medicament for the treatment of a pathological condition or symptom in a mammal, such as a human, which is associated with STAT-6 activation, activation of the IL-4 and/or IL-13 pathways, or p53-induced cellular damage, i.e., with unwanted apoptosis.

The invention also includes a method for binding a compound of formula (I)-(V) to cells and biomolecules comprising IL-4 and/or IL-13 receptors, in vivo or in vitro, comprising contacting said cells or biomolecules with an amount of a compound of formula (I)-(V) effective to bind to said receptors. Cells or biomolecules comprising ligand-bound IL-4/IL-13 receptor sites can be used to measure the selectivity of test compounds for specific receptor subtypes, or can be used as a tool to identify potential therapeutic agents for the treatment of diseases or conditions associated with IL-4/IL-13 pathway activation, by contacting said agents with said ligand-receptor complexes, and measuring the extent of displacement of the ligand and/or binding of the agent, by methods known to the art.

In another embodiment, the present invention provides a compound of formula (I)-(V) that acts to suppress p53 activity in mammalian cells, and a method to effectively suppress p53 activity in the cells of a mammal subject to a stress or pathology that is ameliorated by such suppression. Accordingly, there is provided a method of p53 suppression comprising administering to a mammal in need of said suppression an effective amount of a compound of formula (I)-(V).

The invention also provides novel p53 suppressor compounds, as well as pharmaceutical compositions comprising novel compounds of formula (I)-(V), or a pharmaceutically acceptable salt thereof, in combination with a pharmaceutically acceptable diluent or carrier. Such compounds can be represented by compounds of formula (I), with the proviso that when Y is S, Ar is not phenyl (C₆H₅).

In another embodiment, the present invention provides a compound of formula (I)-(V) that acts to suppress the IL-4/IL-13 pathway in mammalian cells and a method to effectively suppress the IL-4/IL-13 pathway in the cells of a mammal subject to treatment with cancer agents. This suppression enhances the efficacy of and thus, could allow the use of lower doses of the cancer agents, which can reduce side effects from the agents.

In another embodiment, the present invention provides a method for the selection of tumors that respond to antagonism of the IL-4/IL-13 pathways using the compounds of the invention. The method comprises in vitro testing of tissue samples to determine if the IL-4/IL-13 pathway is active by measuring the status of STAT-6 in the cell. The presence of phosphorylated STAT-6 is a marker of an active IL-4/IL-13 pathway (see for example, Takeda, K, et al., “The Essential Role of STAT-6 in IL-4 Signalling”, Nature, 380:627-620,1996. Thus, the method specifically comprises measuring phosphorylated STAT-6 in tissue samples from biopsies or isolated cells using phospho-STAT-6 antibodies (e.g., commercially available antibody 575144 from CalBiochem) by immunohistochemistry or immunoblotting.

Additionally, the invention provides a therapeutic method for preventing or treating a pathological condition or symptom in a mammal, such as a human, wherein the activity of p53 is implicated and antagonism or suppression of its action is desired, comprising administering to a mammal in need of such therapy, an effective amount of a compound of formula (I)-(V), or a pharmaceutically acceptable salt thereof. Such pathological conditions or symptoms include blocking, moderating or reversing the deleterious effects of chemotherapeutic agents, particularly those which damage DNA; radiation, particularly radiation therapy (gamma-, beta- or UV-radiation), ischemic event, including stroke, infarct, ischemia-reperfusion injury and ischemia due to organ, tissue or cell transplantation; environmental pollution or contamination and the like.

The invention also includes a method for binding a compound of formula (I) to cells and biomolecules comprising p53 receptors, in vivo or in vitro, comprising contacting said cells or biomolecules with an amount of a compound of formula (I) effective to bind to said receptors. Cells or biomolecules comprising ligand-bound p53 receptor sites can be used to measure the selectivity of test compounds for specific receptor subtypes, or can be used as a tool to identify potential therapeutic agents for the treatment of diseases or conditions associated with p53 activation, by contacting said agents with said ligand-receptor complexes, and measuring the extent of displacement of the ligand and/or binding of the agent, by methods known to the art.

As used herein, the term “p53” or “p53 activity” refers to p53 protein. The invention is believed to work by temporarily suppressing expression of the p53 gene and/or activity of p53 protein.

### BRIEF DESCRIPTION OF THE FIGURES

FIG. 1 depicts the effects of IBT and PFT-A on B-CLL viability.

FIG. 2 depicts the protective effect of IBT against spontaneous apoptosis and against fludarabine-induced apoptosis.

FIG. 3 shows the ability of the various compounds to block the expression of a STAT-6 dependent reporter gene.

FIG. 4 shows the ability of compounds of the invention to reduce the survival of malignant B cells from a patient with chronic lymphocytic leukemia maintained in tissue culture for 72 hours.

FIG. 5 shows the structures of compounds numbered in FIGS. 3-4. Compound 8 is IBT (control).

FIG. 6 shows the structures of compounds numbered in FIGS. 7-11.

FIG. 7 shows the Inhibition of STAT-6 using compound 8, IBT.

FIGS. 8 and 9 illustrate the Anti-STAT-6 Activity of IBT (compound 8) at several concentrations.

FIG. 10 illustrates a functional measurement of Anti-STAT-6 Activity of compounds of the invention.

FIG. 11 illustrates interferon regulation using IBT.

FIG. 12 illustrates the effect of IBT and IFN on L1236 Cells

### DETAILED DESCRIPTION

The following definitions are used, unless otherwise described: halo is fluoro, chloro, bromo, or iodo. Alkyl, alkoxy, alkenyl, alkynyl, etc. denote both straight and branched groups; but reference to an individual radical such as “propyl” embraces only the straight chain radical, a branched chain isomer such as “isopropyl” being specifically referred to. Aryl denotes a phenyl radical or an ortho-fused bicyclic carbocyclic radical having about nine to ten ring atoms in which at least one ring is aromatic. Heteroaryl encompasses a radical attached via a ring nitrogen or carbon of a monocyclic aromatic ring containing five or six ring atoms consisting of carbon and one to four heteroatoms each selected from the group consisting of non-peroxide oxygen, sulfur, and N(X) wherein X is absent or is H, O, (C₁-C₄)alkyl, phenyl or benzyl. Heteroaryl also includes a radical of an ortho-fused bicyclic heterocycle of about eight to ten ring atoms, particularly a benzo-derivative or one derived by fusing a propylene, trimethylene, or tetramethylene diradical thereto. Preferred heteroaryls include pyridin-4-yl and thiophen-2-yl. The term “heterocyclic ring” “heterocycle,” or “heterocycyl,” is defined as above for formula (I).

It will be appreciated by those skilled in the art that compounds of the invention having a chiral center may exist in and be isolated in optically active and racemic forms. Some compounds may also exhibit polymorphism. It is to be understood that the present invention encompasses any racemic, optically active, polymorphic, or steroisomeric form, or mixtures thereof, of a compound of the invention, which possess the useful properties described herein, it being well known in the art how to prepare optically active forms (for example, by resolution of the racemic form by recrystallization techniques, by synthesis from optically active starting materials, by chiral synthesis, or by chromatographic separation using a chiral stationary phase) and how to determine STAT-6 suppression activity using the standard tests described herein, or using other similar tests which are well known in the art. When R⁴ is OH, enol or keto forms of compounds (II)-(V) are also within the scope of the invention, wherein the adjacent N may be replaced by —N(Rₐ).

Specific and preferred values listed below for radicals, substituents, and ranges, are for illustration only; they do not exclude other defined values or other values within defined ranges for the radicals and substituents.

Specifically, (C₁-C₆)alkyl can be methyl, ethyl, propyl, isopropyl, butyl, isobutyl, sec-butyl, pentyl, 3-pentyl, or hexyl; (C₃-C₇)cycloalkyl can be cyclopropyl, cyclobutyl, cyclopentyl, or cyclohexyl; the term cycloalkyl includes (cycloalkyl)alkyl of the designated number of carbon atoms; (C₁-C₆)alkoxy can be methoxy, ethoxy, propoxy, isopropoxy, butoxy, iso-butoxy, sec-butoxy, pentoxy, 3-pentoxy, or hexyloxy; (C₂-C₆)alkenyl can be vinyl, allyl, 1-propenyl, 2-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, or 5-hexenyl; (C₂-C₆)alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl; (C₂-C₇)alkanoyl can be acetyl, propanoyl or butanoyl; (C₂-C₇)alkanoyloxy can be acetoxy, propanoyloxy, butanoyloxy, isobutanoyloxy, pentanoyloxy, or hexanoyloxy; aryl can be phenyl, indenyl, or naphthyl; and heteroaryl can be furyl, imidazolyl, triazolyl, triazinyl, oxazoyl, isoxazoyl, thiazolyl, isothiazoyl, pyrazolyl, pyrrolyl, pyrazinyl, tetrazolyl, pyridyl, (or its N-oxide), thienyl, pyrimidinyl (or its N-oxide), indolyl, isoquinolyl (or its N-oxide) or quinolyl (or its N-oxide).

A specific value for R¹ and R² is hydroxy, cyano, —N(Rₐ)(R_{b}), —S(Rₐ), —NO₂, (C₂-C₇)alkanoyl, or (C₂-C₇)alkanoyloxy.

A specific value for R¹ and R² together is butylene or benzo.

A specific value for R¹ and R⁴ together is butylene or benzo.

A specific value for R³ is H. A specific value for R⁴ is H.

A specific value for Ar is aryl or heteroaryl, optionally substituted with 1-5, halo, CF₃, hydroxy, CN, —N(Rₐ)(R_{b}), (C₁-C₆)alkyl, (C₁-C₆)alkoxy, (C₂-C₇)alkanoyl, (C₂-C₇)alkanoyloxy, (C₃-C₇)cycloalkyl, (C₂-C₆)alkenyl, or phenyl groups.

Another specific value for Ar is aryl substituted with 1-2, groups. A specific value for Ar is heteroaryl or phenyl substituted with CN, (C₂-C₇)-alkanoyl, (C₂-C₇)alkanolyoxy, (C₂-C₇)cycloalkyl or (C₂-C₆)alkenyl.

A specific value for Ar is phenyl, 2, 3 or 4-pyridyl or 2-thienyl; pyrrolidino, piperidino or morpholino.

A more specific value for Ar is phenyl, 4-pyridyl or 2-thienyl. A specific value for Y is oxy (—O—), S(O)₀₋₂, C(R¹)(R³), N(Rₐ), or —P—.

A specific value for Y is —S—, —O—, —N(Rₐ)—, or —P—.

A specific value for Y is —P—, —Se—, —SO—, —SO₂— or —C(R₁)(R₃)—.

A specific value for Y is —P—, —Se—, —S(O)— or —SO₂—.

A more specific value for Y is —S—, —O—, or —NH—,

A specific value for —N(Rₐ)(R_{b}) is amino.

A specific value for —N(Rₐ)(R_{b}) is pyrrolidino, piperidino or morpholino.

A specific value for halo is Br or F.

A specific method of the invention is treating a disease where the interferon is interferon-α, interferon-β, interferon-γ or a mixture thereof.

Another specific method of the invention is where the interferon is interferon-α.

Another specific method of the invention is where the mammal is a human.

A specific disease for treatment is cancer.

A specific cancer for treatment is leukemia, lymphoma, Hodgkin's disease, lung, head, neck, pancreatic or glioblastoma.

A specific leukemia is chronic lymphocytic leukemia (CLL), acute lymphocytic leukemia (ALL), acute myelogenous leukemia (AML), chronic myelogenous leukemia (CML), or multiple myleoma (MM).

A more specific leukemia is chronic lymphocytic leukemia, or multiple myleoma.

A specific disease for treatment is for a proliferative fibrotic disease.

A specific proliferative fibrotic disease is rheumatoid arthritis, pulmonary fibrosis, interstitial fibrosis of the lung, scleroderma, keloids, renal fibrosis, liver cirrhosis, endometriosis or chronic kidney disease

A specific disease for treatment is for a viral disease.

A specific viral disease is hepatitis, papilloma, semliki forest virus, san angelo virus, punta toro virus, herpes simplex virus, corona virus, cytomegalo virus or Banzi virus.

A specific herpes simplex virus is herpes simplex virus type 1 or herpes simplex virus type 2.

A specific disease for treatment is for an autoimmune disease.

A specific autoimmune disease is lupus erythematosus, multiple sclerosis, infertility from endometriosis, type I diabetes mellitus, Crohn's disease, ulcerative colitis, inflammatory bowel disease, rheumatoid arthritis or pulmonary fibrosis.

A specific method of the invention is where the method comprises the use of a second therapeutic agent.

Another specific method of the invention is where the therapeutic agent is an antiviral agent, an anticancer agent or a therapeutic agent for treatment of autoimmune diseases.

Another specific method of the invention is where the therapeutic agent is ribavirin, acyclovir, valcyclovir or gancyclovir.

A specific method of the invention is administering a mixture of the compounds having formula (I), (II), (III), (IV), (V) or a mixture thereof and interferon delivering a composition to a patient by administering to the patient the composition of the in.

A specific method of the invention is co-administering the compounds having formula (I), (II), (III), (IV), (V) or a mixture thereof simultaneously with the administration of interferon.

A specific method of the invention is administering the compounds having formula (I), (II), (III), (IV), or (V) from 0.1 to about 4 hours prior to the administration of interferon.

A specific method of the invention is administering the compounds having formula (I), (II), (III), (IV), (V) or a mixture thereof from 0.1 to about 4 hours after to the administration of interferon.

The present invention is based on the discovery that PFT-α is both cytotoxic to mammalian cells and unstable in aqueous solution under in vivo conditions. PFT-α undergoes spontaneous ring closure in protic solvents, such as alkanols, to form the imidazo[2,1-b]benzothiazole derivative, abbreviated IBT, as shown in Scheme 1.

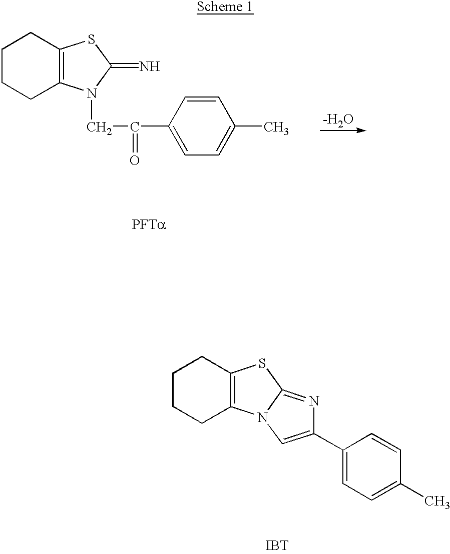

Biological evaluation, described below, demonstrated that IBT is actually responsible for the observed p53 inhibition observed by Komarov et al. (Science, 285, 1733 (1999)). Thus, since IBT and compounds of formula (I) are expected to be both less toxic and more stable than imino compounds such as PFT-α, they are desirable agents for protection of mammalian cells against a wide variety of stressors, including therapeutic agents, and clinical and environmental trauma.

Compounds of formula (I) can be readily prepared as disclosed by Singh et al., Indian J. Chem., 7, 997 (1996), as shown in Scheme 2.

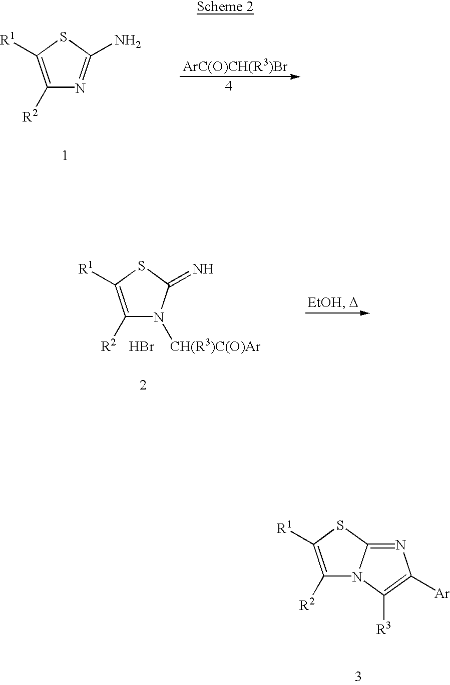

In Scheme 2, a suitable 2-aminobenzothiazole derivative is reacted with an alpha-haloketone in refluxing ethanol resulting in alkylation and ring closure in one single step. An example for the pyridinyl-substituted derivative is given below:

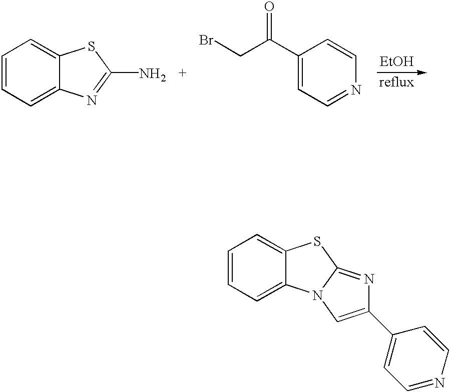

In Scheme 2, the reaction of 1 and 4 can be carried out simply by combining the compounds in a suitable aprotic solvent such as benzene. See, I. Soldabols et al., Khim. Pharm. Zh., 1, 17 (1967). The conversion of 1→3 can also be accomplished in one step by refluxing 1 and the phenacyl bromide 4 in ethanol. Singh et al. used starting materials wherein R¹ and R² together are —(CH₂)₄— or —CH(CH₃)—(CH₂)₃ and Ar is substituted phenyl. Recently, Sumitomo Pharmaceutical Co. Ltd. (Japanese Pat. No. 11-29475) (1999)) disclosed the preparation of certain compounds of formula 2, wherein R³ is H and Ar is substituted phenyl, and Japanese Pat. No. 11-106340 (1999) disclosed the preparation of certain compounds of formula 3 wherein Ar is substituted phenyl or napthyl and R¹ and R² can be, inter alia, H, alkylene or benzo. Compounds of formula 1 were prepared according to Scheme 3.

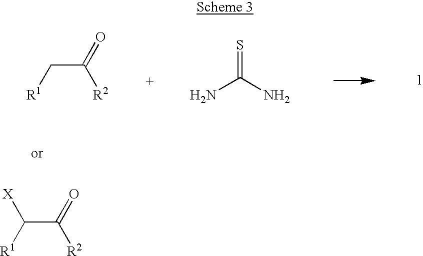

The compounds of formula (I) are disclosed to be useful for “the treatment and prevention of allergic disease and parasitic infectious diseases, or the like.”

Certain of the compounds of formula (I) are useful as intermediates to prepare other compounds of formula (I), as would be recognized by the art.

Compounds of formulae (II)-(V) can be prepared as generally described in PCT/WO97/42192; U.S. Pat. No. 4,020,062, Armianianskii Khim. Zhuv., 43, 245 (1990); Coppola et al., J. Org. Chem., 41, 825 (1976) (II); M. A. Likhale et al., J. Ind. Chem. Soc., 69, 667 (1992); K. T. Potts et al., J. Org. Chem., 35, 3448 (1970); J. E. Francis et al., J. Med. Chem., 34, 281, 2899 (1991) (IV) and A. Guieflier, J. Het. Chem., 27, 421 (1990) (V).

A general method for preparation of imidazo[1,2-a]quinazolines of formula (II) is found in Coppola, et al., wherein a functionalized isatoic anhydride is first alkylated with the alpha-haloketone and then condensed with a suitable thiopseudourea, as shown below for a pyridinyl derivative:

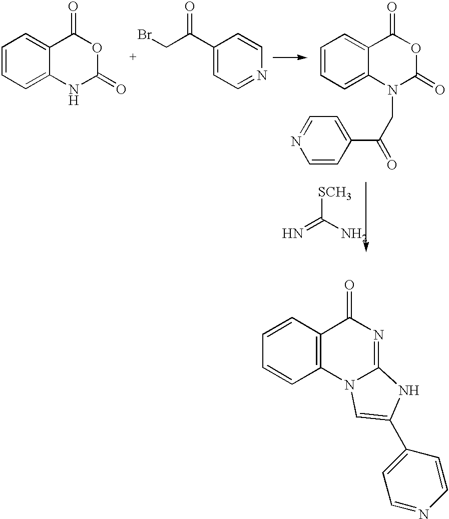

A procedure reported by R. Heckendorn et al., Helv. Chim. Acta, 63, 1 (1980) can be used to prepare the 2-aryl-substituted 1,2,4-triazolo[1,5-a]quinazolines wherein a 2-hydrazinobenzoic acid is condensed with an appropriate N-cyanoimidate ester as shown below:

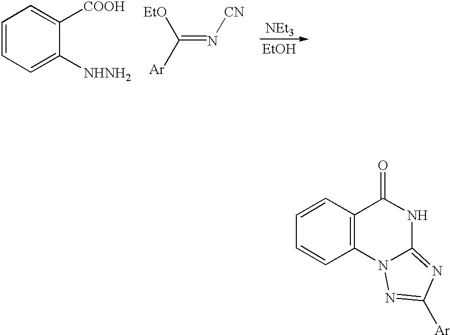

A suitable procedure by Francis, et al., cited above, is used to obtain aryl substituted 1,2,4-triazolo[1,5-c]quinazolines of formula (IV), wherein an appropriate anthranilonitrile is converted to the corresponding carbamate by reaction of the nitrile with ethyl carbonate in the presence of sodium ethoxide, followed by condensation with a suitable aryl carbohydrazide or heteroaryl carbohydrazide as shown below:

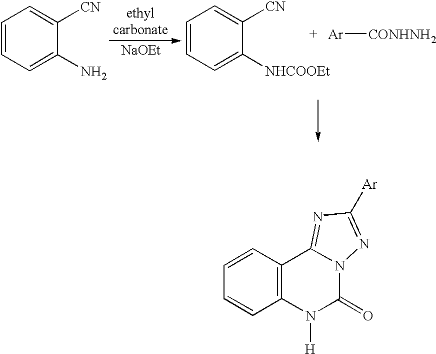

Imidazo[1,2-c]quinazolines of formula (V) may be prepared according to the procedure outlined by Gueffier, et al., wherein a 4-aminoquinazoline is reacted with a bromomethyl aryl ketone in refluxing ethanol. Heteroaryl ketones may also be used as shown below for a pyridinyl derivative:

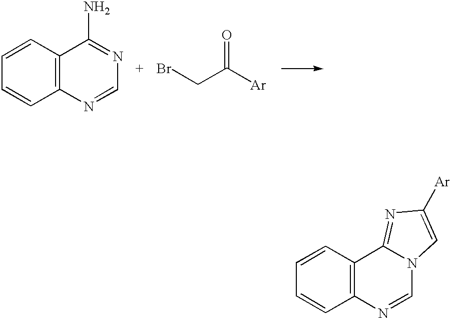

In cases where compounds are sufficiently basic or acidic to form stable nontoxic acid or base salts, administration of the compounds as salts may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

Pharmaceutically acceptable salts may be obtained using standard procedures well known in the art, for example, by reacting a sufficiently basic compound such as an amine with a suitable acid affording a physiologically acceptable anion. Alkali metal (for example, sodium, potassium or lithium) or alkaline earth metal (for example, calcium) salts of carboxylic acids can also be made.

The compounds of formula (I)-(V) can be formulated as pharmaceutical compositions and administered to a mammalian host, such as a human cancer patient, in a variety of forms adapted to the chosen route of administration, i.e., orally or parenterally, by intravenous, intramuscular, topical or subcutaneous routes.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glycerol esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelation.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of formula (I)-(V) to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compounds of formula (I)-(V) can be determined by comparing their in vitro activity, and in vivo activity in animal models. Methods for the extrapolation of effective dosages in mice, and other animals, to humans are known to the art; for example, see U.S. Pat. No. 4,938,949.

Generally, the concentration of the compound(s) of formula (I)-(V) in a liquid composition, such as a lotion, will be from about 0.1-25 wt %, preferably from about 0.5-10 wt %. The concentration in a semi-solid or solid composition such as a gel or a powder will be about 0.1-5 wt %, preferably about 0.5-2.5 wt %.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently administered in unit dosage form, for example, containing 5 to 1000 mg, conveniently 10 to 750 mg, most conveniently, 50 to 500 mg of active ingredient per unit dosage form.

Ideally, the active ingredient should be administered to achieve peak plasma concentrations of the active compound of from about 0.5 to about 75 μM, preferably, about 1 to 50 μM, most preferably, about 2 to about 30 μM. This may be achieved, for example, by the intravenous injection of a 0.05 to 5% solution of the active ingredient, optionally in saline, or orally administered as a bolus containing about 1-100 mg of the active ingredient. Desirable blood levels may be maintained by continuous infusion to provide about 0.01-5.0 mg/kg/hr or by intermittent infusions containing about 0.4-15 mg/kg of the active ingredient(s).

The desired dose may conveniently be presented in a single dose or as divided doses administered at appropriate intervals, for example, as two, three, four or more sub-doses per day. The sub-dose itself may be further divided, e.g., into a number of discrete loosely spaced administrations, such as multiple inhalations from an insufflator or by application of a plurality of drops into the eye.

The ability of a compound of the invention to act as a suppressor of p53 activity may be determined using pharmacological models which are well known to the art, e.g., as disclosed below.

The invention will now be illustrated by the following non-limiting Examples.

### EXAMPLE 1

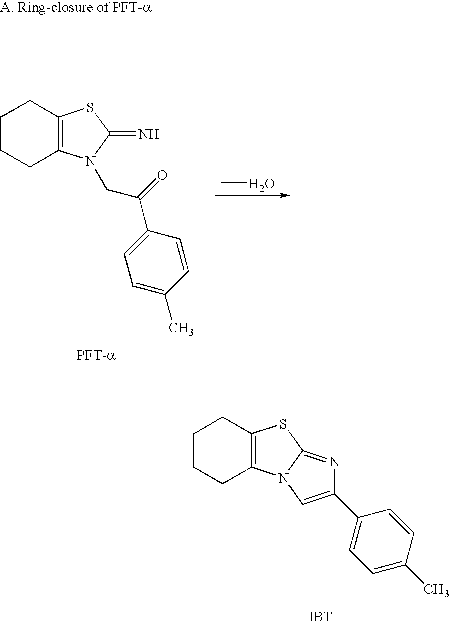

The preparation of PFT-α was accomplished as shown in Scheme 1 by reacting 4-methyl-2-bromoacetophenone with 2-amino-4,5,6,7-tetrahydrobenzothiazole. Upon recrystallization of the PFT-α from isopropyl alcohol, it was noticed that PFT-α readily ring-closed completely to the imidazo[2, 1-b]benzothiazole (IBT). Therefore, a subsequent investigation was undertaken to study the propensity of PFT-α to ring-close in protic solvents. Initial results indicated that PFT-α begins cyclizing at room temperature immediately upon dissolution in protic solvents. Thus, PFT-α was dissolved in DMSO and water dilutions were made from this stock. Reversed phase HPLC analysis of the solution at 25° C. over time gave results as shown in Table 1.

TABLE 1

| Time (h) | % cyclized to IBT |
|---|---|
| 0 | 5 |
| 12 | 47 |
| 24 | 69 |
| 48 | 92 |

In addition, NMR studies were used to confirm the structure of the known IBT and a time course in DMSO-d6 also showed spontaneous conversion of PFT-α to IBT, as judged by the appearance of a new aromatic proton signal at δ 8.50 ppm in the proton spectrum corresponding to the C₃H proton.

### B. 2-(Pyridin-4-yl)imidazo[2,1-b]benzothiazole

A mixture of 2-aminobenzothiazole (0.01 mol) and 4-bromoacetylpyridine (0.01 mol) in anhydrous ethanol (100 mL) is refluxed for 5 hours. The reaction mixture is evaporated to dryness in vacuo and the residue is slurried in ice water. The resulting solid is filtered and dried to provide the title compound as the HBr salt in 60% yield.

### C. 2-(Pyridin-4-yl)imidazo[1,2-a]quinazolin-9-one

Isatoic anhydride (0.01 mol) is treated with sodium hydride (0.012 mol) in dry dimethylacetamide (50 mL) at room temperature for 20 min. and then 4-bromoacetyl-pyridine (0.01 mol) is added and the mixture is stirred at 80 C for 2 hours. The mixture is cooled and poured into cold, aqueous sodium carbonate (500 mL, saturated) and extracted with ethyl acetate (3×200 mL). The organic layer is dried over magnesium sulfate and evaporated to yield the crude alkylated isatoic anhydride which is used directly without further purification for the ring closure procedure. Thus, this ketone intermediate is suspended in acetonitrile (100 mL) containing methyl-2-thiopseudourea (0.012 mol) and sodium carbonate (0.012 mol) and the mixture is refluxed for 30 min. The solvent is then removed in vacuo and replaced with dichloromethane (100 mL). The insoluble salts are filtered off and washed with additional solvent, and the filtrate is evaporated to dryness and diglyme (50 mL) is added to the residue. After addition of one pellet of sodium hydroxide to catalyze the reaction, the mixture is refluxed for 2 hours. Upon cooling, a precipitate forms which is filtered, washed with a small amount of ethyl acetate and recrystallized from methanol or dichloromethane to yield the title compound.

### D. 2-(p-Methylphenyl)[1,2,4]-triazolo[1,5-a]quinazolin-5-4H-one

To a cooled solution (0 C) of N-cyanoarylethylimidate in absolute alcohol (75 mmol in 100 mL EtOH) is added dropwise triethylamine (225 mmol) over 30 min. and then 75 mmol of 2-hydrazinobenzoic acid hydrochloride is added portionwise keeping the temperature below 3 C. The mixture is then allowed to warm slowly to room temperature and is stirred overnight. The resulting mixture is cooled and neutralized with conc. HCl and warmed for 3 hours at 80 C with stirring. The reaction mixture is diluted with water and cooled to 5 C. The resulting solid product which separates is filtered off, washed with cold water, then ether and dried to yield the title compound.

### E. 2-(Pyridin-4-yl)imidazo[1,2-c]quinazoline

A mixture of 4-aminoquinazoline (0.01 mol) and 4-bromoacetylpyridine (0.01 mol) in anhydrous ethanol (100 mL) is refluxed for 5 hours. The reaction mixture is evaporated to dryness in vacuo and the residue is slurried in ice water. The resulting solid is filtered and dried to provide the title compound as the HBr salt.

### F. 2-(Pyridin-4-yl) 1,2,4-triazolo[1,5-c]quinazolin-5(6H)-one

A mixture of the carbamate of anthranilonitrile (prepared by reacting anthranilonitrile (0.21 mol) with ethyl carbonate (250 mL) in absolute ethanol (500 mL) containing sodium ethoxide, 1.67 mol) is reacted with 4-pyridinecarbohydrazide (one to one equivalence, 55 mmol each) in 2-ethoxyethanol (185 mL) containing tri-n-propylamine (7.4 mL) by heating at reflux for 16 h, cooling, and treating with water gradually to promote crystallization. After overnight refrigeration, the solid product is collected and recrystallized from ethanol.

### EXAMPLE 2

### Effect of the p53 Inhibitory Compounds on B-CLL Viability

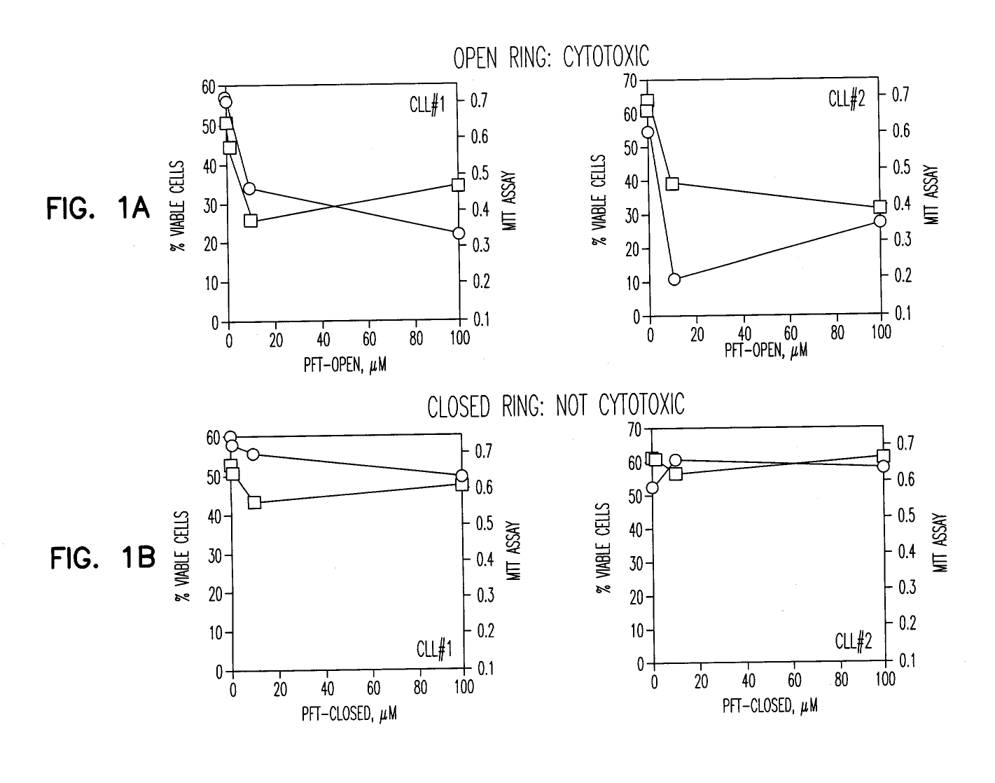

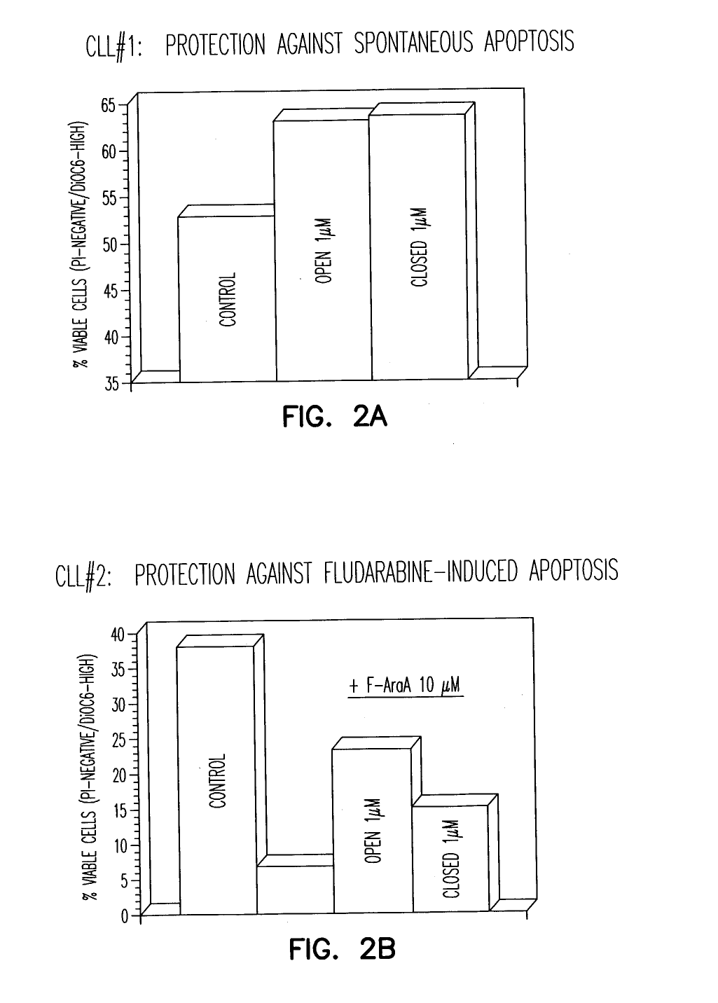

The malignant lymphocytes from two patients with chronic lymphocytic leukemia [CLL] were isolated by ficoll-hypaque sedimentation and suspended at a density of 1 million cells per milliliter in RPMI 1640 medium supplemented with 10% fetal bovine serum. Two hundred microliter aliquots of cells were dispersed in the wells of culture plates containing the indicated final concentrations of either PFT-α (“PFT-open”) or IBT (PFT-closed). After 3 days culture, viable cells were enumerated by fluorescence-activated cell sorting [FACS] after staining with propidium iodide [PI]. Viable cells excluded the dye [open circles]. In addition, cell metabolism was assessed by the ability of the cells to exclude the tetrazolium dye MTT [closed squares]. As shown in FIG. 1, the PFT-open dose-dependently reduced CLL survival, whereas PFT-closed [i.e., IBT] was non-toxic at concentrations up to 100 micromolar.

### EXAMPLE 3

### Protection Against Spontaneous Apoptosis and Apoptosis Induced by the Anti-metabolite Fludarabine

Chronic lymphocytic leukemia [CLL] cells were cultured for 3 days as described in Example 2. Some of the cultures were supplemented with one micromolar of PFT-open or PFT-closed, as indicated. In the experiment shown in the bottom panel of FIG. 2, some of the cultures also contained the cytotoxic adenine nucleoside analog fludarabine [abbreviated F-AraA]. Fludarabine is the first line treatment for CLL, and the toxicity of the drug is dependent upon the p53 pathway. To assess healthy, viable cells, staining was done with both PI, as indicated in Example 2, and with the mitochondrial dye DiOC6. Cells that were both PI negative and DIOC6 high were enumerated by FACS. While PFT-α and IBT exhibited nearly equivalent effects on untreated CLL cells, IBT exerted less protective effects when combined with CLL cells treated with F-AraA than did PFT-α.

### EXAMPLE 4

### Screening of Compounds of Formula (I) for Inhibition of IL-4 Transcriptional Activity

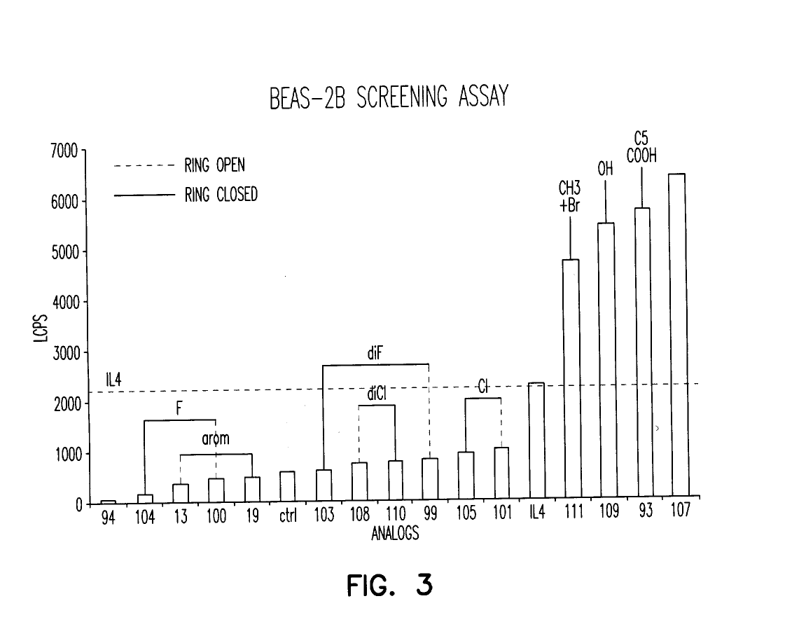

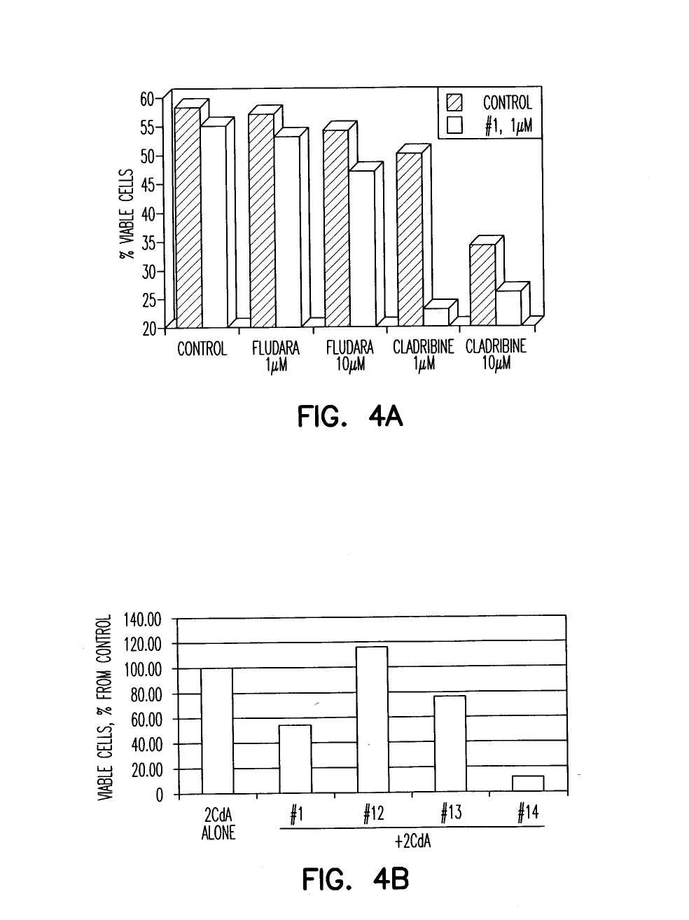

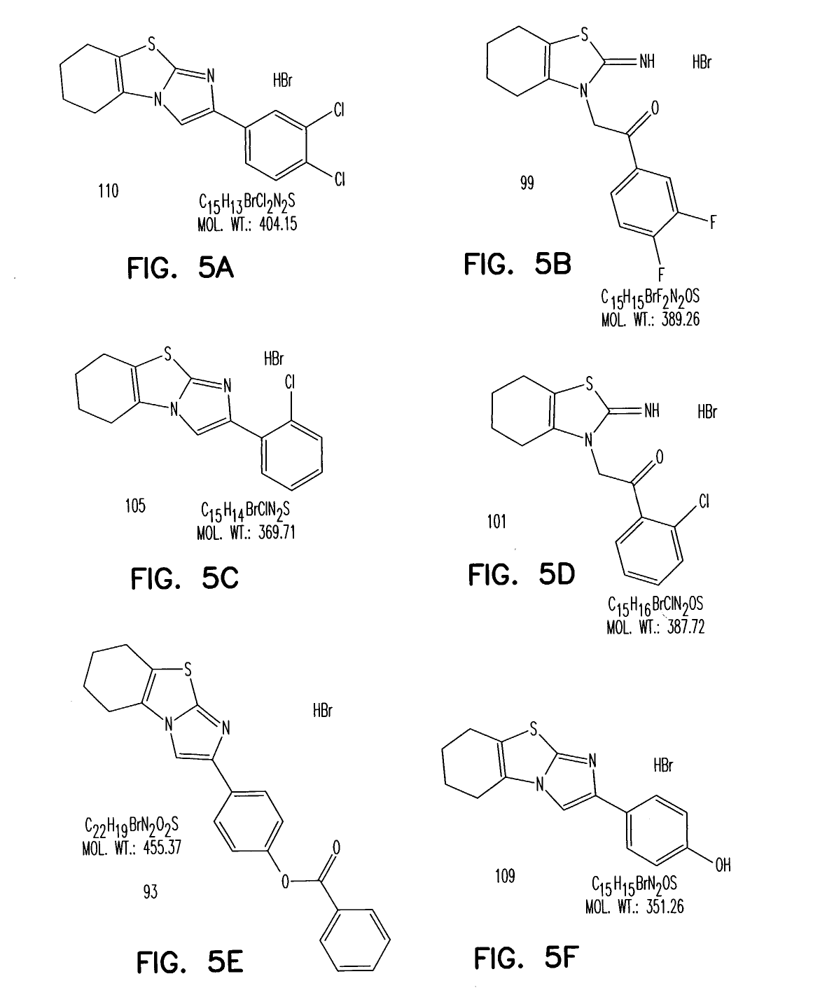

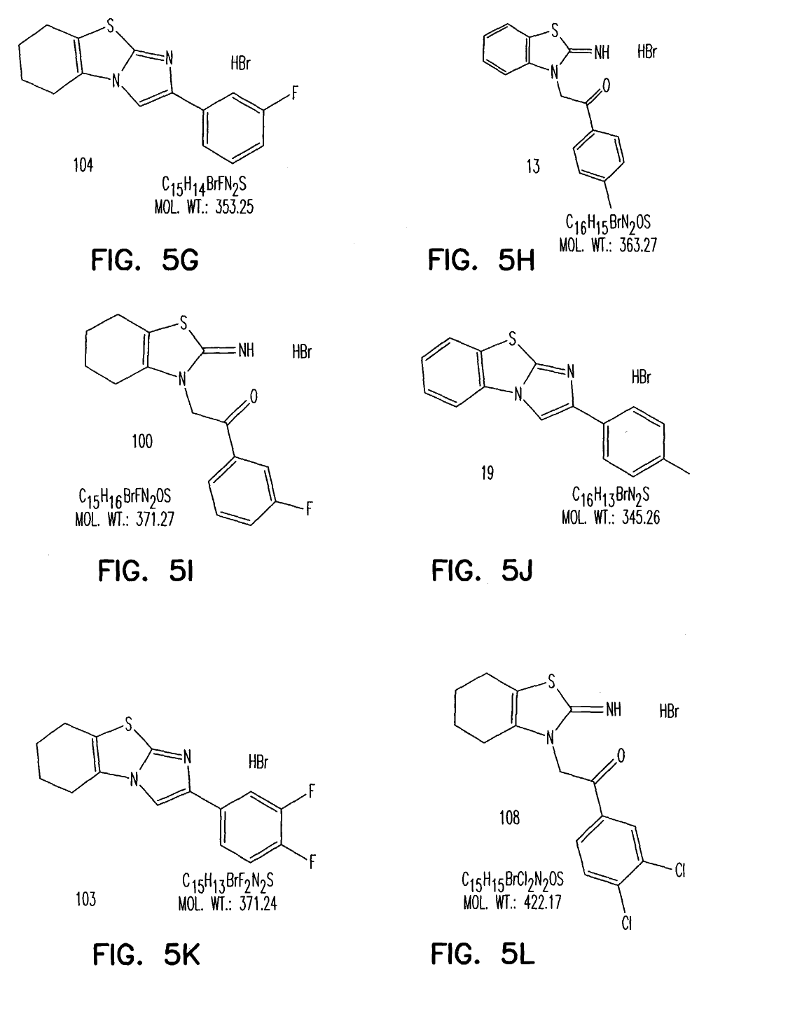

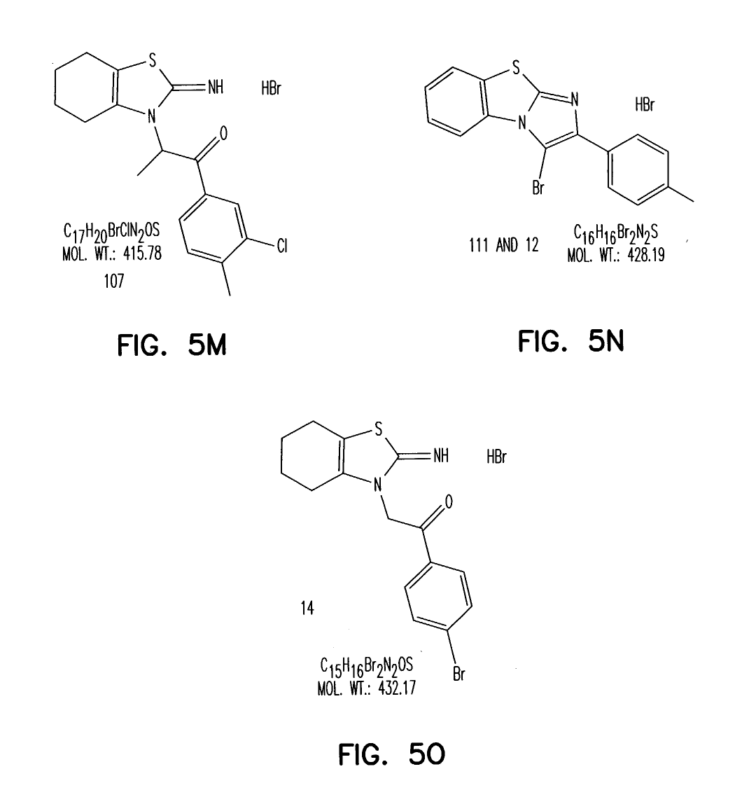

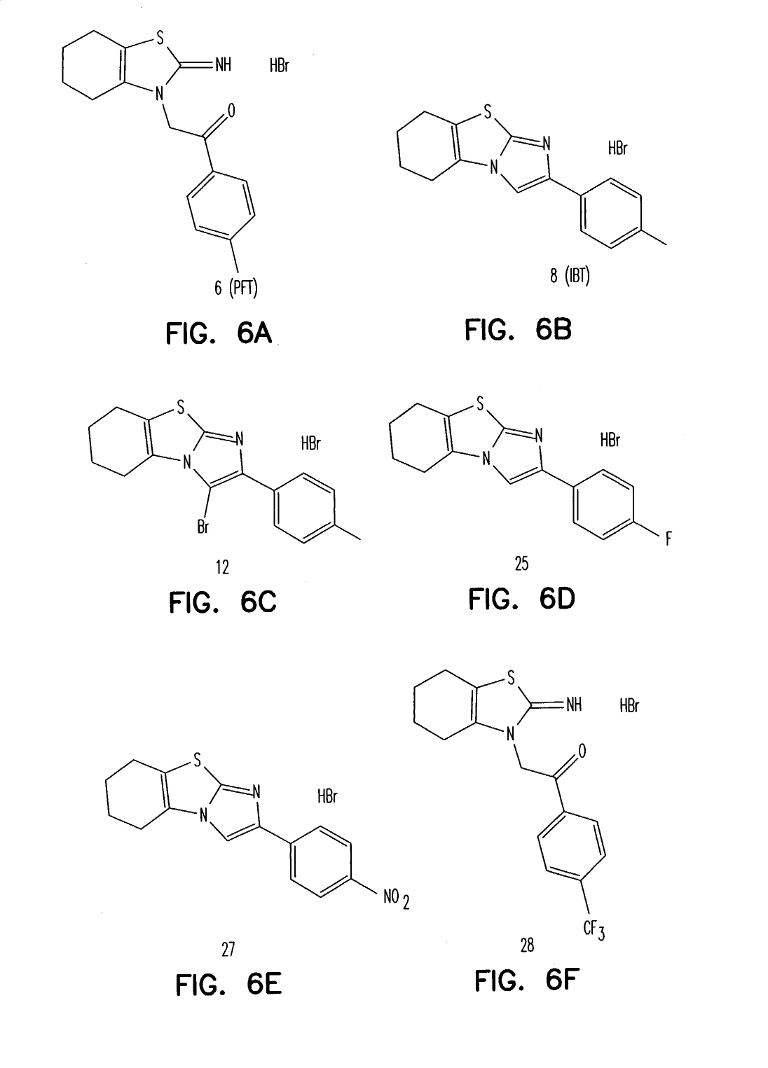

The BEAS-2B human airway epithelial cells were transiently transfected with the human 12/15-lipoxygenase promoter/luciferase reporter gene. Cells were then incubated with the IBT analogs (FIG. 5) at 10 μM for 1 hour, followed by IL-4 (10 ng/ml). After 16 hours, luciferase was measured using a chemiluminometer. The STAT-6 induction was normalized using the B-gal results as “background.” The viability of the treated cells was visually verified at the end of the incubation, and found to be >95%. Results shown in FIG. 3 are the mean of duplicate measurements.

### EXAMPLE 5

### Sensitization of CLL Cells to Apoptosis by IL-4/IL-13 Antagonists

Chronic lymphocytic leukemia (CLL) cells were isolated from whole blood of patients, cultured in RPMI-1640 supplemented with 10% FB. CLL cells were pre-incubated for 1 hour with the indicated analogs (FIG. 5) at 1 μM and exposed for 24 hours to the nucleoside analogs Fludarabine (Fludara) and Cladribine (2 CdA) at 1 and 10 μM. Cells were then incubated for 10 minutes in growing medium with 5 μg/ml Propidium iodide and 40 nM DiOC₆ and analyzed by flow cytometry. Viable cells (Y axis) and high DiOC₆ (FL-1) and low PI (FL-3) fluorescence.

### EXAMPLE 6

### Preparation of Pharmaceutical Dosage Forms

The following illustrate representative pharmaceutical dosage forms, containing a compound of formula (I)-(V), for therapeutic or prophylactic use in humans.

TABLE 1

| (i) | mg/tablet |
|---|---|
| Compound of Formula (I)-(V) | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |

TABLE 2

| (ii) | mg/tablet |
|---|---|
| Compound of Formula (I)-(V) | 20.0 |
| Microcrystalline cellulose | 410.0 |
| Starch | 50.0 |
| Sodium starch glycolate | 15.0 |
| Magnesium stearate | 5.0 |
| | 500.0 |
| (iii) Capsule | mg/capsule |
| Compound of Formula (I)-(V) | 10.0 |
| Colloidal silicon dioxide | 1.5 |
| Lactose | 465.5 |
| Pregelatinized starch | 120.0 |
| Magnesium stearate | 3.0 |
| | 600.0 |
| (iv) Injection 1 (1 mg/ml) | mg/ml |
| Compound of Formula (I)-(V) | 1.0 |
| Dibasic sodium phosphate | 12.0 |
| Monobasic sodium phosphate | 0.7 |
| Sodium chloride | 4.5 |
| 01 N Sodium hydroxide solution | q.s. |
| (pH adjustment to 7.0-7.5) | |
| Water for injection | q.s. ad 1 mL |
| (v) Injection 2 (10 mg/ml) | mg/ml |
| Compound of Formula (I)-(V) | 10.0 |
| Monobasic sodium phosphate | 0.3 |
| Dibasic sodium phosphate | 1.1 |
| Polyethylene glycol 400 | 200.0 |
| 01 N Sodium hydroxide solution | q.s. |
| (pH adjustment to 7.0-7.5) | |
| Water for injection | q.s. ad 1 mL |
| (vi) Aerosol | mg/can |
| Compound of Formula (I)-(V) | 20.0 |
| Oleic acid | 10.0 |
| Trichioromonofluoromethane | 5,000.0 |
| Dichlorodifluoromethane | 10,000.0 |
| Dichlorotetrafluoroethane | 5,000.0 |

The above formulations may be obtained by conventional procedures well known in the pharmaceutical art.

### EXAMPLE 7

### Inhibition of STAT-6

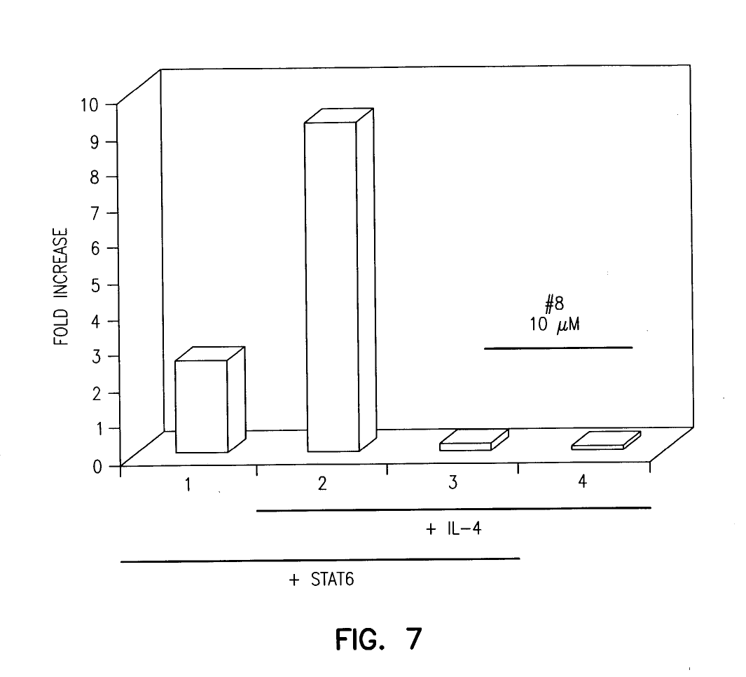

Human airway epithelial cells, BEAS-2B, were transiently transfected with the human 12/15-lipoxygenase promoter/luciferase reporter plasmid with and without a STAT6 expressing plasmid. The cells were incubated with 10 μM of STAT-6 (Control Sample 1); 10 μM STAT-6 (Sample 2); a mixture of STAT-6 and compound 8 (Sample 3); and compound 8 alone (Sample 4); for 1 hour. This was followed treatment with 10 ng/mL of IL-4 (except control). After 16 hours, luciferase was measured using a chemiluminometer. The STAT-6 induction was normalized using the β-gal results as “background”. The viability of the treated cells was visually verified at the end of the incubation, and found to be>95%. The results are illustrated in FIG. 7.

### EXAMPLE 8

### Measurement of Anti-STAT-6 Activity of IBT (Compound 8)

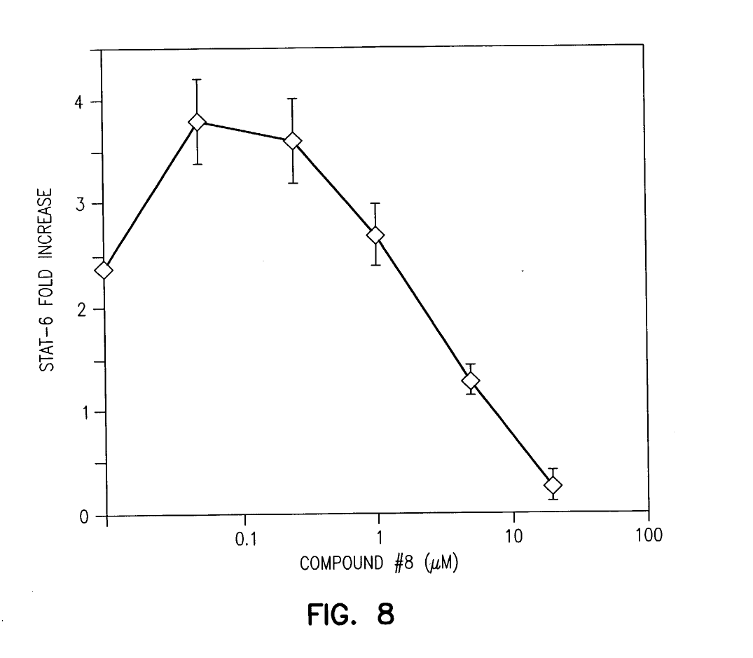

Human airway epithelial cells, BEAS-2B, were transiently transfected with the human 12/15-lipoxygenase promoter/luciferase reporter gene. The cells were incubated with compound 8 at concentrations of 0.075 μM, 0.5 μM, 1.0 μM, 7.5 μM and 40 μM for 1 hour. The treatment was followed by incubation with IL-4 (10 ng/mL). After 16 hours, luciferase was measured using a chemiluminometer. The STAT-6 induction was normalized using the β-gal results as “background”. The results are the mean of triplicate measurments, all standard deviations were lower than 8%. The results are illustrated in FIG. 8.

### EXAMPLE 9

### Measurement of Anti-STAT-6 Activity

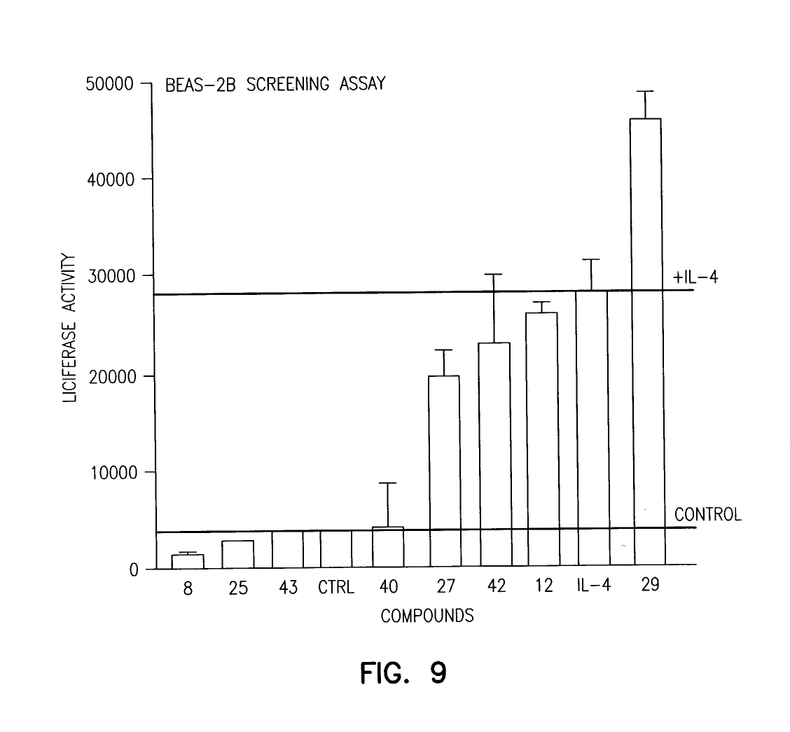

The inhibitory activity of the compounds of the invention was tested at a fixed concentration of 10 μM in IL-4 stimulated BEAS-2B human airway cells using the 12/15-lipoxygenase promoter/luciferase as described in the previous Examples. The results are illustrated in FIG. 9.

### EXAMPLE 10

### Functional Measurement of Anti-STAT-6 Activity

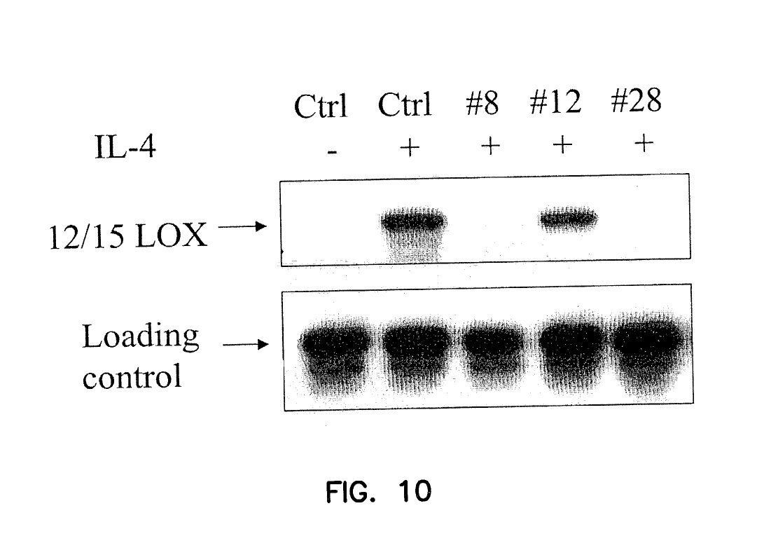

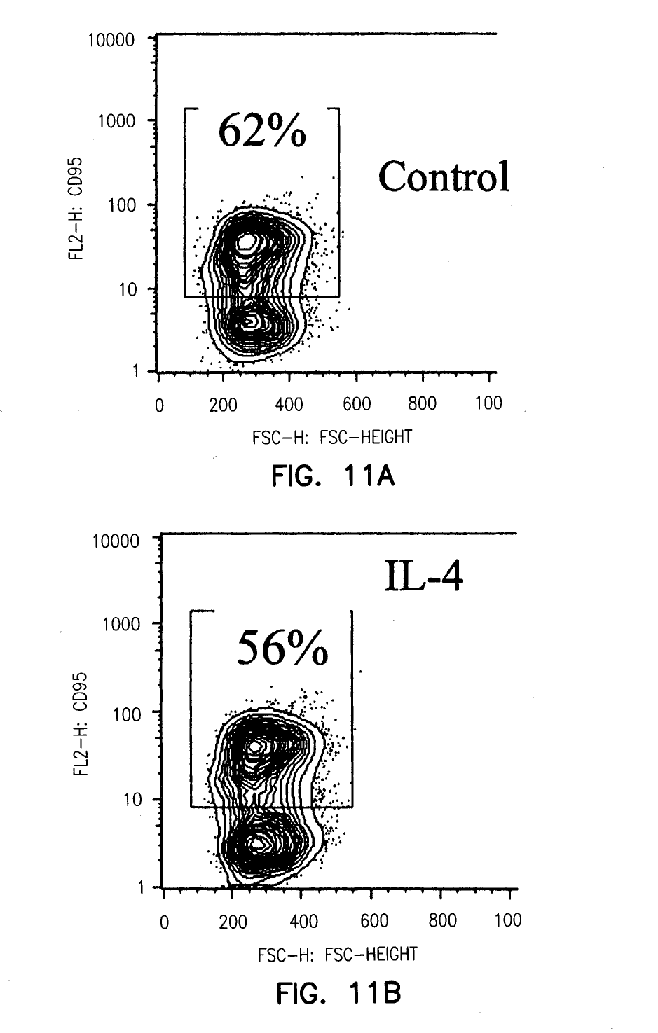

The effectiveness of compounds 8, 12 and 28 on the IL-4-induced expression of the protein 12/15 lipooxygenase (12/15-LOX) was measured in primary human monocytes. The cells were pre-incubated for 1 hour with 10 μM of the test compound. This was followed by exposure to 10 ng/ml of IL-4. The expression of 12/15-LOX was quantified by immunoblotting using a specific monoclonal antibody and compared to a loading control. The results are illustrated in FIG. 10.

### EXAMPLE 11

### Interferon Regulation Using IBT (Compound 8)

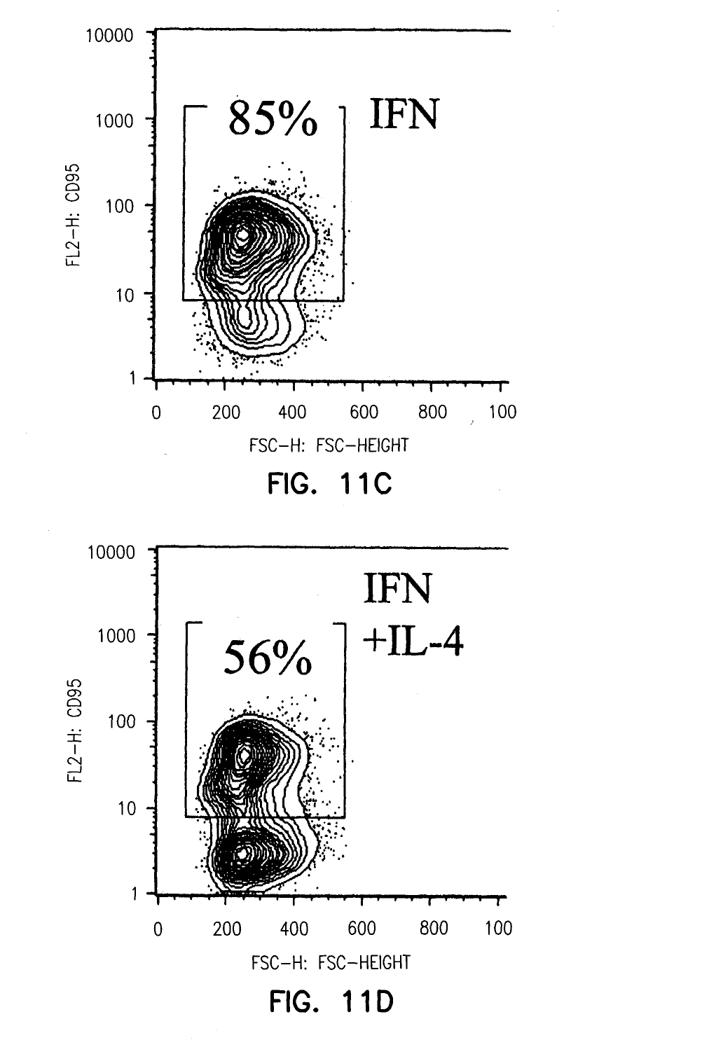

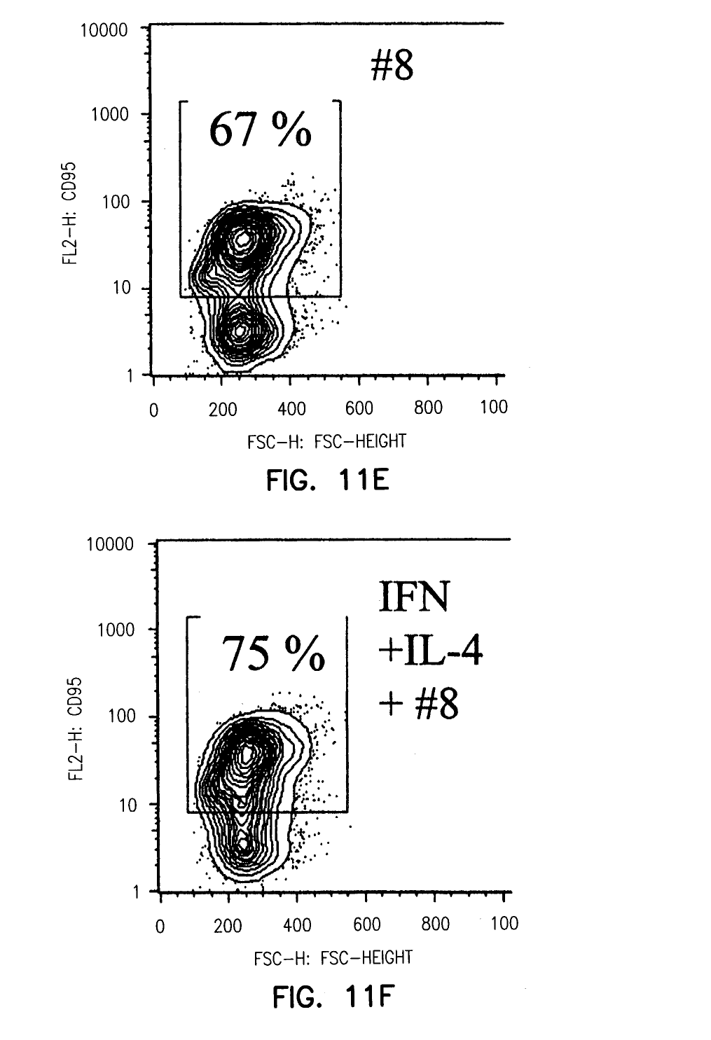

The B cells RAMOS were incubated with IL-4 (FIG. 11B); leukocyte-purified interferons (IFN, 10⁴ U/ml) (FIG. 11C); INF AND IL-4 (FIG. 11D); compound 8 alone (FIG. 11E); and leukocyte-purified interferons (IFN, 10⁴ U/ml) (FIG. 11E); IL-4 (10 ng/ml) and compound #8 (10 μM) (FIG. 11F); for 48 hrs. The control is illustrated in FIG. 11A. The surface expression of the Fas receptor (also known as FasR or CD95) was then measured by flow cytometry using a PE-conjugated monoclonal antibody. The results as a percentage of CD95+ cells are indicated in each panel in FIG. 11.

### EXAMPLE 12

### Effect of IBT, and Interferon on Hodgkin's Disease

Human Hodgkin's lymphoma cells L1236 were incubated for 3, 5 or 7 days in complete RPMI1640 and 10% FBS. The cells were plated at 3×10⁵ cells/mL. These cells (Li236) are similarly to other Hodgkin's cell lines and other tumor types such as pancreatic cancers, which express high levels of IL-4 and/or IL-13 receptors or have been shown to require IL-13 for their survival.

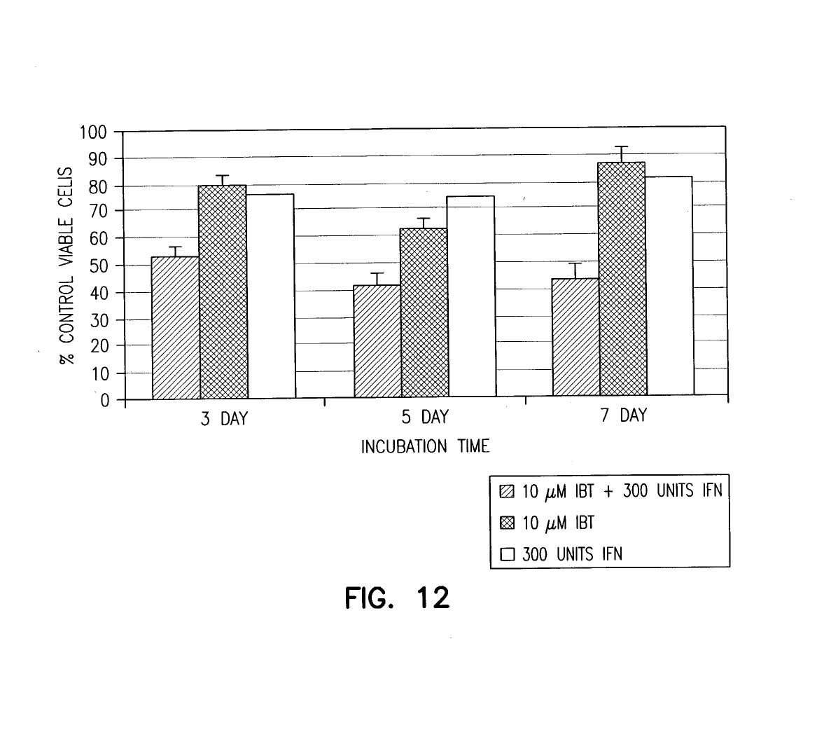

The L1236 cells were cultured in presence of type 1 interferon (leukocyte interferon, primarily IFN-α), or in presence of IBT alone at doses up to 10 μM, only a small anti-proliferative effect was observed (FIG. 12, second and third columns). When the L1236 cells were cultured in presence of interferon and IBT combined (FIG. 12, first column), a stronger growth-inhibitory activity was observed. The effect was more pronounced after 7 days of incubation. These results indicate that IBT or related molecules by inhibiting that the IL4 and/or IL-13 signaling can enhance the effects of interferons. These results suggest that IBT or related analogs will be clinically useful in combination with interferons in diseases where interferons have been shown to be useful such as, but not limited to, cancers and viral infections.

The results are illustrated in FIG. 12. The y-axis represent the normalized viability when compared to control cells incubated with 0.1% DMSO with no interferon or IBT added, adjusted at 100%. The cell viability was determined using CellTiter 96 Aqueous One Solution Cell Proliferation Assay (Promega). (CellTiter 96 uses a novel tetrazolium compound (MTS) and an electron coupling reagent, phenazine ethosulfate.)

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A therapeutic method to enhance the efficacy of interferon treatment comprising administering to a mammal subject to interferon treatment a compound which is an antagonist of the IL-4 or IL-13 signal transduction pathway in an amount effective to enhance said efficacy.

2. The method of claim 1, wherein the interferon is interferon-α interferon-β, interferon-γ or a mixture thereof.

3. The method of claim 1, wherein the interferon is interferon-α.

4. The method of claim 1, wherein the mammal is a human.

5. The method of claim 1, wherein the interferon treatment is for cancer.

6. The method of claim 5, wherein the cancer is leukemia, lymphoma, Hodgkin's disease, lung, head, neck, pancreatic or glioblastoma.

7. The method of claim 6, wherein the leukemia is cronic lymphocytic leukemia, acute lymphocytic leukemia, acute myelogenous leukemia, cronic myelogenous leukemia, or multiple myleoma.

8. The method of claim 7, wherein the leukemia is cronic lymphocytic leukemia, or multiple myleoma.

9. The method of claim 1, wherein the interferon treatment is for a proliferative fibrotic disease.

10. The method of claim 9, wherein the proliferative fibrotic disease is rheumatoid arthritis, pulmonary fibrosis, interstitial fibrosis of the lung, scleroderma, keloids, renal fibrosis, liver cirrhosis, or chronic kidney disease.

11. The method of claim 1, wherein the interferon treatment is for a viral disease.

12. The method of claim 11, wherein the viral disease is hepatitis, papilloma, semliki forest virus, san angelo virus, punta toro virus, herpes simplex virus, corona virus, cytomegalo virus or banzi virus.

13. The method of claim 1, wherein the interferon treatment is for an autoimmune disease.

14. The method of claim 13, wherein the autoimmune disease is lupus erythematosus, multiple sclerosis, infertility from endometriosis, type I diabetes mellitus, Crohn's disease, ulcerative colitis, inflammatory bowel disease, rheumatoid arthritis or pulmonary fibrosis.

15. The method of claim 14, further comprising the use of a second therapeutic agent.

16. The method of claim 15, wherein the second therapeutic agent is an antiviral agent, an anticancer agent or a therapeutic agent for treatment of autoimmune diseases.

17. The method of claim 16, where the therapeutic agent is ribavirin, acyclovir, valcyclovir or gancyclovir.

18. The method of claim 1, wherein the antagonist of the IL-4 or IL-13 signal transduction pathway is a compound of formula (I): 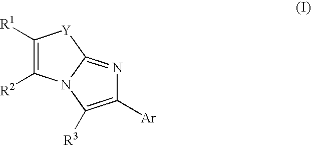
wherein R¹, R² and R³ are independently hydrogen, halo, hydroxy, cyano, —N(Rₐ)(R_{b}), —S(Rₐ), —NO₂, (C₁-C₆)alkyl, (C₁-C₆)alkoxy, (C₂-C₆)alkynyl, (C₂-C₆)alkenyl, (C₂-C₇)alkanoyl, (C₂-C₇)alkanoyloxy, or (C₃-C₇)cycloalkyl or R¹ and R² taken together are benzo, optionally substituted by R¹, (C₃-C₅)alkylene or methylene dioxy; wherein Rₐ and R_{b} are each independently hydrogen, (C₁-C₃)alkyl, (C₂-C₄)alkanoyl, phenyl, benzyl, or phenethyl; or Rₐ and R_{b} together with the nitrogen to which they are attached are a 5-6 membered heterocyclic ring;
Ar is aryl, heteroaryl, or a 5-6 membered heterocyclic ring, optionally comprising 1-3 N(Rₐ), nonperoxide O or S atoms, optionally substituted with 1-5 groups where the groups are selected from halo, CF₃, hydroxy, CN, —N(Rₐ)(R_{b}), (C₁-C₆)alkyl, (C₁-C₆)alkoxy, (C₂-C₇)alkanoyl, (C₂-C₇)alkanoyloxy, (C₃-C₇)cycloalkyl, (C₂-C₆)alkenyl, and phenyl;
Y is oxy (—O—), —S(O)₀₋₂—, Se, —C(R¹)(R³)—, —N(Rₐ)—, or —P—,
or a pharmaceutically acceptable salt thereof.

19. The method of claim 18, wherein the heterocyclic ring, is pyrrolidino, piperidino or morpholino.

20. The method of claim 18, wherein the aryl, heteroaryl or heterocyclic group is substituted with 1 or 2 groups.

21. The method of claim 18, wherein R¹ and R² together is butylene or benzo.

22. The method of claim 18, wherein R³ is hydrogen.

23. The method of claim 18, wherein Ar is phenyl, 4-pyridyl or 2-thienyl.

24. The method of claim 18, wherein Ar is a 5-6 membered heterocyclic ring, comprising 1-3 N(Rₐ), nonperoxide O or S atoms.

25. The method of claim 18, wherein Ar is pyrrolidino, piperidino or morpholino.

26. The method of claim 18, wherein Y is —O—(oxy), —S(O)₀₋₂—, —C(R¹)(R³)—, —NR¹, or —P—.

27. The method of claim 18, wherein Y is —O—, —N(Rₐ)— or —P—.

28. The method of claim 18, wherein Y is S.

29. The method of claim 18, wherein —N(Rₐ)(R_{b}) is amino.

30. The method of claim 18, wherein halo is Br or F.

31. The method of claim 18, wherein —N(Rₐ)(R_{b}) is pyrrolidino, piperidino or morpholino.

32. The method of claim 1, wherein the antagonist of the IL-4 or IL-13 signal transduction pathway is a compound of formula (II): 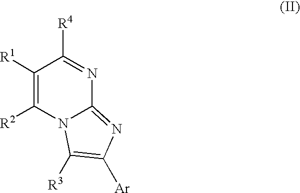
wherein R¹, R², R³ and R⁴ are independently hydrogen, halo, hydroxy, cyano, —N(Rₐ)(R_{b}), —S(Rₐ), —NO₂, (C₁-C₆)alkyl, (C₁-C₆)alkoxy, (C₂-C₆)-alkynyl, (C₂-C₆)alkenyl, (C₂-C₇)alkanoyl, (C₂-C₇)alkanoyloxy, or (C₃-C₇)cycloalkyl or R¹ and R² taken together are benzo, optionally substituted by R¹, (C₃-C₅)alkylene or methylene dioxy; wherein Rₐ and R_{b} are each independently hydrogen, (C₁-C₃)alkyl, (C₂-C₄)alkanoyl, phenyl, benzyl, or phenethyl; or Rₐ and R_{b} together with the nitrogen to which they are attached are a 5-6 membered heterocyclic ring, or R¹ and R⁴ together with the atoms to which they are attached are benzo, (C₃-C₅)alkylidene or methylenedioxy;
Ar is aryl, heteroaryl, or a 5-6 membered heterocyclic ring, optionally comprising 1-3 N(Rₐ), nonperoxide O or S atoms, optionally substituted with 1-5 groups where the groups are selected from halo, CF₃, hydroxy, CN, —N(Rₐ)(R_{b}), (C₁-C₆)alkyl, (C₁-C₆)alkoxy, (C₂-C₇)alkanoyl, (C₂-C₇)alkanoyloxy, (C₃-C₇)cycloalkyl, (C₂-C₆)alkenyl, and phenyl;
or a pharmaceutically acceptable salt thereof.

33. The method of claim 32, wherein the heterocyclic ring, is pyrrolidino, piperidino or morpholino.

34. The method of claim 32, wherein the aryl, heteroaryl or heterocyclic group is substituted with 1-2 groups.

35. The method of claim 32, wherein N(Rₐ)(R_{b}) is pyrrolidino, piperidino or morpholino.

36. The method of claim 32, wherein the compound of formula (II) has formula (IIa) or (IIb): 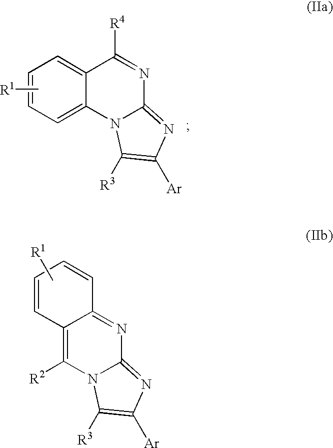
or a pharmaceutically acceptable salt thereof.

37. The method of claim 1, wherein the antagonist of the IL-4 or IL-13 signal transduction pathway is a compound of formula (III): 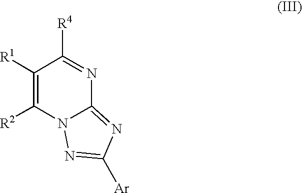
wherein R¹, R², and R⁴ are independently hydrogen, halo, hydroxy, cyano, N(Rₐ)(R_{b}), S(Rₐ), NO₂, (C₁-C₆)alkyl, (C₁-C₆)alkoxy, (C₂-C₆)alkynyl, (C₂-C₆)alkenyl, (C₂-C₇)alkanoyl, (C₂-C₇)alkanoyloxy, or (C₃-C₇)cycloalkyl or R¹ and R² taken together are benzo, optionally substituted by R, (C₃-C₅)alkylene or methylene dioxy; wherein Rₐ and R_{b} are each independently hydrogen, (C₁-C₃)alkyl, (C₂-C₄)alkanoyl, phenyl, benzyl, or phenethyl; or Rₐ and R_{b} together with the nitrogen to which they are attached are a 5-6 membered heterocyclic ring, or R¹ and R⁴ together with the atoms to which they are attached are benzo, (C₃-C₅)alkylidene or methylenedioxy;
Ar is aryl, heteroaryl, or a 5-6 membered heterocyclic ring, optionally comprising 1-3 N(Rₐ), nonperoxide O or S atoms, optionally substituted with 1-5 groups where the groups are selected from halo, CF₃, hydroxy, CN, —N(Rₐ)(R_{b}), (C₁-C₆)alkyl, (C₁-C₆)alkoxy, (C₂-C₇)alkanoyl, (C₂-C₇)alkanoyloxy, (C₃-C₇)cycloalkyl, (C₂-C₆)alkenyl, and phenyl;
or a pharmaceutically acceptable salt thereof.

38. The method of claim 37, wherein the heterocyclic ring, is pyrrolidino, piperidino or morpholino.

39. The method of claim 37, wherein the aryl, heteroaryl or heterocyclic group is substituted with 1-2 groups.

40. The method of claim 1, wherein the antagonist of the IL-4 or IL-13 signal transduction pathway is a compound of formula (IV): 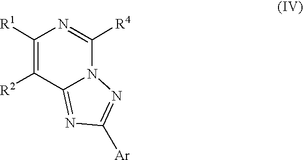
wherein R¹, R² and R⁴ are independently hydrogen, halo, hydroxy, cyano, N(Rₐ)(R_{b}), S(Rₐ), NO₂, (C₁-C₆)alkyl, (C₁-C₆)alkoxy, (C₂-C₆)alkynyl, (C₂-C₆)alkenyl, (C₂-C₇)alkanoyl, (C₂-C₇)alkanoyloxy, or (C₃-C₇)cycloalkyl or R¹ and R² taken together are benzo, optionally substituted by R¹, (C₃-C₇)cycloalkyl or methylene dioxy; wherein Rₐ and R_{b} are each independently hydrogen, (C₁-C₃)alkyl, (C₂-C₄)alkanoyl, phenyl, benzyl, or phenethyl; or Rₐ and R_{b} together with the nitrogen to which they are attached are a 5-6 membered heterocyclic ring;
Ar is aryl, heteroaryl, or a 5-6 membered heterocyclic ring, optionally comprising 1-3 N(Rₐ), nonperoxide O or S atoms, optionally substituted with 1-5 groups where the groups are selected from halo, CF₃, hydroxy, CN, —N(Rₐ)(R_{b}), (C₁-C₆)alkyl, (C₁-C₆)alkoxy, (C₂-C₇)alkanoyl, (C₂-C₇)alkanoyloxy, (C₃-C₇)cycloalkyl, (C₂-C₆)alkenyl, and phenyl;
or a pharmaceutically acceptable salt thereof.

41. The method of claim 40, wherein the heterocyclic ring, is pyrrolidino, piperidino or morpholino.

42. The method of claim 40, wherein the aryl, heteroaryl or heterocyclic group is substituted with 1-2 groups.

43. The method of claim 1, wherein the antagonist of the IL-4 or IL-13 signal transduction pathway is a compound of formula (V): 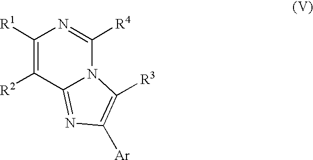
wherein R¹, R², R³ and R⁴ are independently hydrogen, halo, hydroxy, cyano, N(Rₐ)(R_{b}), S(Rₐ), NO₂, (C₁-C₆)alkyl, (C₁-C₆)alkoxy, (C₂-C₆)-alkynyl, (C₂-C₆)alkenyl, (C₂-C₇)alkanoyl, (C₂-C₇)alkanoyloxy, or (C₃-C₇)-cycloalkyl or R¹ and R² taken together are benzo, optionally substituted by R¹, (C₃-C₅)alkylene or methylene dioxy; wherein Rₐ and R_{b} are each independently hydrogen, (C₁-C₃)alkyl, (C₂-C₄)alkanoyl, phenyl, benzyl, or phenethyl; or Rₐ and R_{b} together with the nitrogen to which they are attached are a 5-6 membered heterocyclic ring;
Ar is aryl, heteroaryl, or a 5-6 membered heterocyclic ring, optionally comprising 1-3 N(Rₐ), nonperoxide O or S atoms, optionally substituted with 1-5 groups where the groups are selected from halo, CF₃, hydroxy, CN, —N(Rₐ)(R_{b}), (C₁-C₆)alkyl, (C₁-C₆)alkoxy, (C₂-C₇)alkanoyl, (C₂-C₇)alkanoyloxy, (C₃-C₇)cycloalkyl, (C₂-C₆)alkenyl, and phenyl;
or a pharmaceutically acceptable salt thereof.

44. The method of claim 43, wherein the heterocyclic ring, is pyrrolidino, piperidino or morpholino.

45. The method of claim 43, wherein the aryl, heteroaryl or heterocyclic group is substituted with 1-2 groups.

46. The method of claim 1, wherein the compound of formula (I) is administered w of a compound of formula (I):
